(19) United States
(12) Patent Application Publication
(10) Pub. No.: US 2024/0368654 A1
(43) Pub. Date: Nov. 7, 2024

(54) APPARATUS AND METHOD FOR CONTINUOUS PRODUCTION OF RNA

(71) Applicant: UNIVERSITY OF MASSACHUSETTS, Boston, MA (US)

(72) Inventor: Carl Lawton, Lowell, MA (US)

(21) Appl. No.: 18/561,036

(22) PCT Filed: Jun. 7, 2022
(86) PCT No.: PCT/US2022/032436

§ 371 Date: Nov. 15, 2023

Related U.S. Application Data
(60) Provisional application No. 63/197,551, filed on Jun. 7, 2021.

(51) Int. Cl. C12P 19/34; C12N 15/10

(52) U.S. Cl. CPC C12P 19/34; C12N 15/101

(57) ABSTRACT

A modular apparatus for continuous production of purified RNA, the apparatus including, in operable communication: a reaction module including a bioreactor configured for continuous synthesis of an affinity-tagged RNA molecule by in vitro transcription using reactants in an in vitro transcription mixture; a control module including a sensor; and an RNA separation system configured to receive a transcription reaction product comprising the affinity-tagged RNA molecule from the bioreactor and to separate the affinity tagged RNA molecule from the transcription reaction product, wherein the RNA separation system includes a capture module including at least one affinity column for binding the affinity-tagged RNA molecule and a purification module including a continuous chromatography column, wherein the control module controls input of the reactants to the bioreactor and monitors separation of the affinity-tagged RNA molecule using the sensor, and wherein the sensor is in communication with the reaction module and the RNA separation system and the sensor provides feedback control of the transcription mixture in the reaction module and the transcription reaction product in the RNA separation system.

### CROSS-REFERENCE TO RELATED APPLICATIONS

This application claims priority to U.S. Provisional Application 63/197,551 filed on Jun. 7, 2021, which is incorporated herein by reference in its entirety.

### BACKGROUND

In vitro mRNA synthesis is a breakthrough platform technology brought to the forefront with unprecedented success by the current FDA approved, mRNA vaccines directed against severe acute respiratory syndrome virus coronavirus 2 (SARS COV-2), which is the coronavirus that causes the respiratory disease coronavirus disease 19 (COVID-19). Unfortunately, more than 550,000 Americans, as well as millions globally, have died waiting for the approved vaccine. Although the mRNA vaccines appear to be stemming the tide for now, the real danger is that if significant strides are not made in stemming the surge both in the U.S. as well as globally, variants of SARS COV-2 have the potential to affect humankind with ever increasing lethal ferocity. To ensure that this does not happen, global availability of mRNA vaccines is needed. This is currently not possible due to two major impediments. First, the cost of the mRNA vaccine is over $20 per dose, which is prohibitive for developing countries and needs to be in line with other vaccines which are an order of magnitude less. Second, the ultra-cold chain limitations associated with storage of mRNA vaccines needs to be eliminated because this infrastructure is not available in undeveloped countries.

The present disclosure addresses the major bottlenecks impacting the widespread use of mRNA vaccines by providing a continuous manufacturing technology through which low cost, mRNA vaccines can be produced. Through use of the disclosed methods and systems, formulations can be made available globally and at a lower cost.

### BRIEF DESCRIPTION

Described herein is a modular apparatus for continuous production of purified RNA, the apparatus comprising, in operable communication: a reaction module comprising a bioreactor configured for continuous synthesis of an affinity-tagged RNA molecule by in vitro transcription using reactants in an in vitro transcription mixture; a control module comprising a sensor; and an RNA separation system configured to receive a transcription reaction product comprising the affinity-tagged RNA molecule from the bioreactor and to separate the affinity tagged RNA molecule from the transcription reaction product, wherein the RNA separation system comprises a capture module comprising at least one affinity column for binding the affinity-tagged RNA molecule and a purification module comprising a continuous chromatography column, wherein the control module controls input of the reactants to the bioreactor and monitors separation of the affinity-tagged RNA molecule using the sensor, and wherein the sensor is in communication with the reaction module and the RNA separation system and the sensor provides feedback control of the transcription mixture in the reaction module and the transcription reaction product in the RNA separation system.

Also described herein is a method for continuous production of purified RNA, the method comprising: contacting a DNA template comprising a sequence encoding an open reading frame and a sequence encoding an affinity tag with reactants in an in vitro transcription mixture, and under conditions suitable to provide a transcription reaction product comprising an affinity-tagged RNA molecule, wherein the contacting is performed in a bioreactor operating in continuous mode; feeding the transcription reaction product to an affinity column comprising an immobilized ligand and binding the affinity-tagged RNA molecule to the immobilized ligand; eluting the bound affinity-tagged RNA molecule from the affinity column; passing the eluted affinity-tagged RNA molecule through a continuous chromatography column to provide the purified RNA; and providing feedback control of the in vitro transcription mixture in the bioreactor and the transcription reaction product in the affinity column.

The above described and other features are exemplified by the following figures and detailed description.

### BRIEF DESCRIPTION OF THE DRAWINGS

The following figures are exemplary embodiments wherein the like elements are numbered alike.

FIG. 1 is an illustration of a system for the continuous automated manufacture of purified mRNA.

FIG. 2 is an illustration of an ELISA-based solid state polyadenine binding protein (PABP) sensor.

FIG. 3 is an illustration of a PABP protein bound to a silicon nanowire (SiNW) anchored on a surface of a substrate.

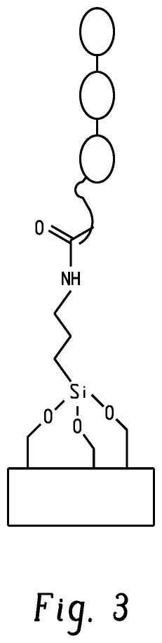

FIG. 4 shows a PAGE analysis of polyadenine binding protein PABP expression.

FIG. 5 shows a PAGE analysis purification of PABP conjugated to Fractogel® EMD synthetic methacrylate based polymeric beads.

FIG. 6 shows purification of mRNA according to the present disclosure.

### DETAILED DESCRIPTION

The present disclosure provides a compact and modular apparatus for the continuous and automated manufacture of purified RNA. The apparatus is readily and easily transportable (mobile) and has a relatively small footprint. The disclosed apparatus helps to prevent potential pandemic outbreaks by mobilizing vaccine producing units (e.g., equipment and personnel) to new outbreak areas caused by new viral strains or mutations. The compact, modular equipment can be air-lifted to any area of the world to set-up mobile production. In addition, the apparatus can be regionally distributed across the United States, or in other countries, allowing for localized production and the elimination of potential bottlenecks (e.g., failure in a centralized manufacturing plant which takes down the entire manufacturing system).

Described herein also are methods for the continuous and automated manufacture of RNA utilizing the disclosed apparatus.

In the present disclosure, synthesis of an RNA molecule of a given sequence is performed at a large scale. The term “large scale” refers to a reaction yield of the mRNA molecule in the order of milligram quantities, preferably of at least one gram.

The apparatus and methods described herein provide a method for the continuous production of RNA. The volume of RNA produced can be on a manufacturing scale or on a smaller scale such as lab scale. A “manufacturing scale” process is capable of producing large volumes and amounts of a preparation containing the RNA of interest and yielding amounts of the RNA of interest that meet the demands for clinical trials as well as for market supply.

The term “gene” refers to a nucleotide sequence associated with a biological function. Thus, a gene includes a coding sequence and/or the regulatory sequence required for its expression. A gene can also include nonexpressed DNA segments that, for example, form recognition sequences for other proteins. A gene can be obtained from a variety of sources, including cloning from a source of interest or synthesizing from known or predicted sequence information, and may include sequences designed to have desired parameters.

A ribonucleic acid (RNA) is a nucleic acid molecule composed of adenosine-monophosphate, uridine-monophosphate, guanosine-monophosphate and cytidine-monophosphate monomers which are connected to each other along a backbone formed by phosphodiester bonds between the sugar of a first monomer and a phosphate moiety of a second, adjacent monomer. The RNA comprises a messenger RNA (mRNA), a small interfering RNA (siRNA), a transfer RNA (tRNA), a ribosomal RNA (rRNA), a small nuclear RNA (snRNA), a microRNA (miRNA), a transfer-messenger RNA, a non-coding RNA, or a combination thereof. In an aspect, the RNA is an mRNA.

A messenger RNA (mRNA) is a coding RNA molecule, and is a functional RNA molecule which can be translated into a peptide or protein. The mRNA molecule can encode one (monocistronic), two (bicistronic), or more (multicistronic) open reading frames (ORF). The mRNA comprises an ORF encoding a protein. The mRNA may include a cap structure at a 5′ end, a poly A tail at a 3′ end, or a combination thereof.

An open reading frame (ORF) is a sequence of several nucleotide triplets which may be translated into a peptide or protein. An ORF is a nucleotide sequence consisting of a number of nucleotides that may be divided by three, which starts with a start codon (e.g. ATG or AUG) and which terminates with a stop codon (e.g., TAA, TGA, or TAG or UAA, UAG, UGA, respectively). The open reading frame may be isolated or it may be incorporated in a longer nucleic acid sequence, for example in a vector or an mRNA. An ORF may also be referred to herein as “protein coding region” or “coding region”.

A nucleotide sequence is “operably linked” when placed into a functional relationship with another nucleotide sequence. For example, a nucleotide sequence for a promoter is operably linked to a coding sequence if it stimulates the transcription of the sequence. In general, nucleotide sequences that are operably linked are contiguous, and in the case of a signal sequence both contiguous and in reading phase.

The term “promoter” refers to a minimal nucleotide sequence which is sufficient to direct transcription of a particular gene. The promoter includes the core promoter, which is the minimal portion of the promoter required to properly initiate transcription and can also include regulatory elements that promote transcription. A promoter can be constitutive or inducible. A constitutive promoter refers to one that is always active and/or constantly directs transcription of a gene above a basal level of transcription. An inducible promoter is one which is capable of being induced by an added molecule or factor.

As used herein “transcription mixture” or “in vitro transcription mixture” refers to the combination of materials (e.g., RNA polymerase, ribonucleoside triphosphates, buffer, magnesium ions, etc.) needed to facilitate in vitro transcription of a linear DNA template.

A “transcription reaction product” refers to the product produced by an in vitro transcription reaction. The transcription reaction product can include at least one RNA of interest as a desired reaction product, an unreacted reactant, and/or a reaction byproduct, e.g., double stranded RNA (dsRNA), pyrophosphate, and the like.

Disclosed herein is a modular apparatus for continuous production of purified RNA, the apparatus comprising: a reaction module comprising a bioreactor configured for continuous synthesis of an affinity-tagged RNA molecule by in vitro transcription using reactants in an in vitro transcription mixture; a control module comprising a sensor; and an RNA separation system configured to receive a transcription reaction product comprising the affinity-tagged RNA molecule from the bioreactor and to separate the affinity-tagged RNA molecule from the at least one transcription reaction product, wherein the RNA separation system comprises a capture module comprising at least one affinity column for binding the affinity-tagged RNA molecule and a purification module comprising a continuous chromatography column, wherein the control module controls input of the reactants to the bioreactor and monitors separation of the mRNA using the sensor, and wherein the sensor is in communication with the bioreactor and the mRNA separation system and the sensor provides feedback control of the transcription mixture in the reaction module and the transcription reaction product in the RNA separation system.

In an aspect of the described methods, the RNA molecule comprises a messenger RNA (mRNA) molecule. In an aspect, the apparatus is a modular and compact system designed for the continuous production and purification of mRNA from a DNA sequence encoding a protein of interest.

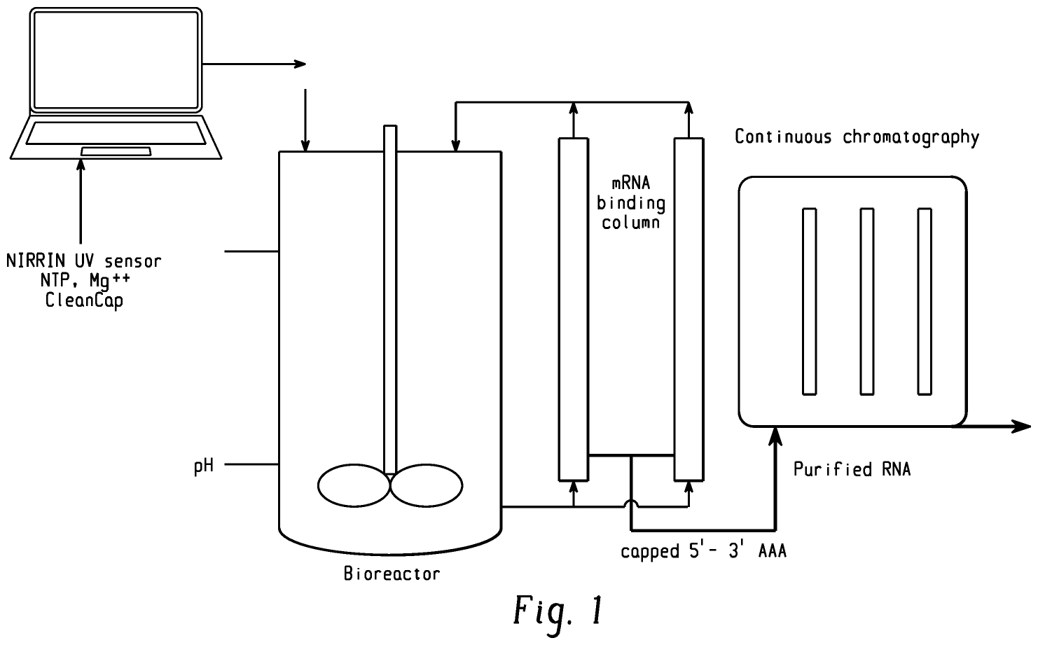

An exemplary apparatus is illustrated in FIG. 1. The apparatus is composed of a reaction module, a control module, and a mRNA separation system including a capture module and a purification module. The reaction module, the control module, and the mRNA separation system are in operable communication with one another.

The bioreactor operates in a continuous mode, and is configured for continuous synthesis of an RNA molecule by in vitro transcription using reactants in an in vitro transcription mixture. The bioreactor includes at least one inlet connected to a supply line that feeds (supplies) the reactants for in vitro transcription to the bioreactor. The supply line is also connected to a reservoir containing the reactant. The bioreactor can include a single inlet connected to a single supply line and a corresponding reservoir. Alternatively, the bioreactor can include a plurality of inlets, each connected to its own supply line, and each supply line is connected to a separate reservoir containing a different reactant for the in vitro transcription mixture. In an aspect, the apparatus includes a plurality of supply lines each of which independently supplies a reactant for in vitro transcription to the bioreactor. The apparatus further includes at least one pump for feeding a reactant from the reservoir through the supply line to the interior of the bioreactor. In addition, the bioreactor includes an outlet from which the transcription reaction product prepared by in vitro transcription in the bioreactor, is transported to the mRNA separation system.

The control module comprises at least one sensor. In an aspect, the control module comprises a plurality of sensors. The sensor is in communication with the reaction module and the mRNA separation system, and provides feedback control of the transcription mixture in the reaction module as well as feedback control of the transcription reaction product in the RNA separation system. In an aspect, the sensor comprises a Raman-based biochemical sensor, an at-line photo-electric ELISA solid state sensor, an inline ultraviolet or near infrared sensor (UV or NIR) sensor (NIRRIN Technologies), a Fournier near-infrared (FT-NIR) sensor, or a combination thereof.

In an aspect, at least one sensor provides feedback control of the transcription mixture in the reaction module. The control module receives and analyzes data received from the at least one sensor. The sensor detects the concentration of each of the reactants in the in vitro transcription mixture in the bioreactor, and the control module controls the input of the reactants to the bioreactor based on the data received from the sensor. In an aspect, the sensor monitors the concentration of at least one reactant in the in vitro transcription mixture and the control module determines whether the concentration of the reactant in the in vitro transcription mixture is above, at, or below a threshold level based on the data received from the sensor. In an aspect, the sensor comprises a plurality of sensors that independently monitor nucleotide triphosphates, capping molecules, RNA polymerase, buffer, magnesium levels, or a combination thereof in the transcription mixture. When the concentration of the reactant falls below the threshold level, the reactant is supplied to the bioreactor.

In an aspect, a sensor in the control module provides feedback control and monitors the transcription reaction product in the RNA separation system. The control module also monitors and controls separation of the RNA molecule in the RNA separation system. For example, through signals received from the sensor, the control module monitors the RNA separation system to determine when maximum binding capacity of the affinity-tagged RNA molecule in the affinity column is reached. When the maximum binding capacity of the affinity-tagged RNA molecule in the affinity column is detected by the sensor, the control module instructs the elution of the RNA molecule from the affinity column. The control module thus controls and instructs the equilibrating, eluting, and washing of the affinity column(s).

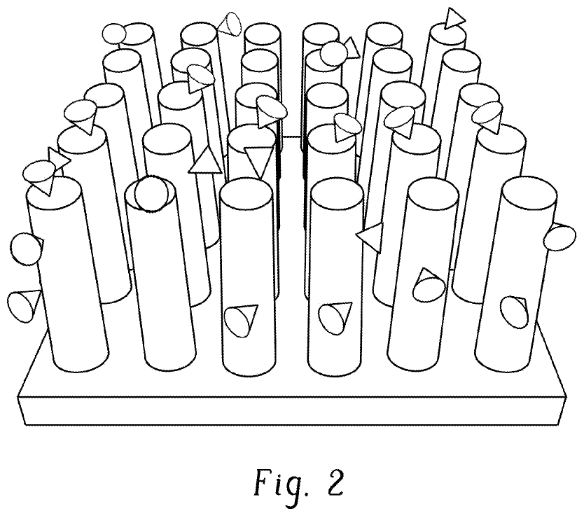

In an aspect, the sensor in communication with the RNA separation system is an at-line photo-electric ELISA solid state sensor that is capable of measuring binding of the affinity-tagged RNA molecule to the ligand in the affinity column. FIG. 2A shows silicon nanowires conjugated with PABP and bound to a substrate. FIG. 2B is an exploded view of the outlined area in FIG. 2A. Binding of affinity-tagged RNA molecules having an poly(A) tail to the PABP conjugated to the silicon nanowire conjugate elicits a signal in the sensor.

The transcription reaction product is continuously fed from the capture module to the RNA separation system where the RNA is separated from the transcription reaction product and purified. The RNA separation system is composed of a capture module and a purification module. The capture module includes at least one affinity chromatography column (“affinity column”). The RNA separation system includes an inlet through which the transcription reaction product flows from the reaction module to the RNA separation system. In particular, the transcription reaction product flows from the outlet of the bioreactor to an inlet of the capture module and from an outlet of the capture module to an inlet of the purification module. A first transfer line connects the outlet of the bioreactor to an inlet of the capture module. A second transfer line connects the outlet of the capture module to the inlet of the purification module.

In FIG. 1, the capture module includes a first affinity column and a second affinity column. The number of affinity columns in the capture module can be at least two, at least three, or at least four. The number of affinity columns is not necessarily limited and can be modified as needed to facilitate the continuous flow of the transcription reaction product through the capture module. Determination of the number of affinity columns can be based upon the volume of the reaction mixture in the bioreactor, the volume of reactants flowing from the bioreactor into the capture module, and the time needed for binding, eluting, washing, and equilibrating separate affinity columns.

In an aspect, the capture module includes a first affinity column and a second affinity column, each including an inlet and an outlet. In an aspect, the outlet of the first affinity column and the outlet from the second affinity column are separately connected to the second transfer line, which connects to the inlet of the purification module. In an aspect, the capture module comprises a plurality of affinity columns each including an inlet and an outlet, and the outlet of each of the plurality of affinity columns is connected to the second transfer line.

In an aspect, the transcription reaction product comprises an affinity-tagged RNA molecule and the affinity column comprises an immobilized ligand which binds the affinity-tagged RNA molecule present in the transcription reaction product. In an aspect, the affinity column comprises an immobilized poly(A)-binding protein and the affinity tag is a poly-A tag. In another aspect, the the affinity column comprises immobilized poly(histidine) PSBP and the affinity tag is a poly(histidine) tail.

The purification module includes a continuous chromatography column having an inlet and an outlet. In an aspect, the continuous chromatography column is a continuous flow chromatography column. The transcription reaction product flows from the capture module to the inlet of the continuous chromatography column. In the continuous chromatography column, the RNA molecule is separated from any remaining contaminants present in the transcription reaction product following passage through the capture module. In an aspect, the flow path through the apparatus includes flow of the transcription reaction product from the outlet of the bioreactor to an inlet of the capture module, through the capture module to an outlet of the capture module, from the outlet of the capture module to an inlet of the purification module, through the purification module to an outlet of the purification module, and through the outlet of the purification module.

The apparatus described herein can be used in a method for the continuous production of purified RNA. Described herein is a method for continuous production of purified mRNA comprising contacting a DNA template comprising a sequence encoding an open reading frame and a sequence encoding an affinity tag with reactants in an in vitro transcription mixture, and under conditions suitable to provide an in vitro transcription reaction product comprising an affinity-tagged RNA molecule, wherein the contacting is performed in a bioreactor operating in continuous mode; feeding the transcription reaction product to at least one affinity column comprising an immobilized ligand and binding the affinity-tagged RNA molecule to the immobilized ligand; eluting the bound affinity-tagged RNA molecule from the at least one affinity column; passing the eluted affinity-tagged RNA molecule through a continuous chromatography column to provide the purified RNA, and providing feedback control of the in vitro transcription mixture in the bioreactor and the transcription reaction product in the affinity column. In an aspect, the RNA molecule is an mRNA molecule.

In the context of the methods and apparatus described in this disclosure, the contacting and synthesis of the RNA molecule are performed in the bioreactor, the binding of the synthesized RNA molecule and the eluting of the bound RNA are performed in the capture module, and the passing of the eluted RNA through the continuous chromatography column is performed in the purification module. In the disclosed method(s), each of the described contacting, adding, binding, eluting, passing, and providing steps are continuous processes.

In an aspect, a DNA template is contacted with an in vitro transcription mixture (transcription mixture) under conditions suitable to provide an in vitro transcription reaction product comprising an affinity-tagged RNA molecule. The contacting includes adding the DNA template and the transcription mixture to the bioreactor of the reaction module. The DNA template comprises a promoter suitable for in vitro transcription operably linked to a sequence encoding an open reading frame and a sequence encoding an affinity tag. The DNA template may further encode a termination signal for in vitro transcription. Specifically, the open reading frame corresponds to a desired RNA molecule to be prepared. The RNA molecule is prepared by in vitro transcription of the portion of the DNA template encoding the open reading frame. In an aspect, the RNA molecule is synthesized in the bioreactor by continuously contacting the DNA template with the transcription reaction mixture under conditions suitable to synthesize the mRNA transcript. In an aspect, the affinity tag comprises a polyadenylate tail (poly(A) tail), a poly(histidine) tail, or a combination thereof.

The DNA template can be a component present in the transcription reaction mixture or the DNA template can be immobilized on a substrate. The immobilization of the DNA template allows the repeated use of the template and reduces contamination of the RNA molecule by residual DNA. The DNA template can be a chemically synthesized DNA molecule, an isolated DNA restriction fragment, a plasmid DNA, or an amplified DNA molecule (e.g., a complementary deoxyribonucleic acid (cDNA) prepared by PCR). The DNA template can be a double-stranded duplex or a unit that comprises a double-stranded promoter region upstream of a single-stranded RNA coding region. The DNA template may be modified to facilitate immobilization to a solid support at the 5′ end, the 3′ end, or at an internal nucleotide, for example, by attaching a ligand to the DNA template. In an aspect, the DNA template is a linearized DNA sequence encoding a promoter sequence having a high binding affinity for its respective RNA polymerase and operably linked to the sequence encoding the open reading frame.

The transcription mixture includes the ribonucleoside triphosphates (NTPs) needed to synthesize the RNA molecule. The NTPs can be modified or unmodified. modified ribonucleotide triphosphates, or a combination thereof. In an aspect the NTPs include guanosine triphosphate (GTP), adenosine triphosphate (ATP), cytidine triphosphate (CTP), uridine triphosphate (UTP), pseudouridine triphosphate, dihydrouridine triphosphate, 4-thiouridine triphosphate, inosine triphosphate, a methylguanosine triphosphate (e.g., 7′-methylguanosine triphosphate, 2,7-dimethylguanosine triphosphate, 2,2,7-trimethylguanosine triphosphate), queuosine triphosphate, or a combination thereof. The fraction of each of the NTPs based on the total amount of NTPs is optimized based on the fraction of the respective NTP in the corresponding RNA molecule and the type and amount of DNA template. The transcription mixture also includes a buffer, an RNA polymerase, Mg⁺⁺ ions, and a capping molecule (e.g., a capping enzyme). In an aspect, the in vitro transcription mixture comprises guanosine-5′-triphosphate, adenosine triphosphate, cytidine triphosphate, uridine triphosphate, pseudouridine triphosphate, dihydrouridine triphosphate, 4-thiouridine, inosine triphosphate, 7-methylguanosine triphosphate, 2,7-dimethylguanosine triphosphate, 2,2,7-trimethylguanosine triphosphate, queuosine triphosphate, a capping reagent, magnesium ions, RNA polymerase, a buffer, or a combination thereof. The transcription mixture is prepared by combining the four NTPs, the buffer, the RNA polymerase, the Mg⁺⁺ ions, and the capping molecules.

In an aspect, the transcription reaction mixture comprises a buffer, for example, 4-(2-hydroxyethyl)-1-piperazineethanesulfonic acid (HEPES), tris(hydroxymethyl) aminomethane (Tris), or a combination thereof. The pH value of the buffer can be adjusted as needed using, for example, using NaOH, KOH or HCl. In an aspect, the buffer has a pH of 6 to 8.5, 6.5 to 8.0, 6.8 to 7.8, 6.8 to 7.5, or 7.0 to 7.5.

In an aspect, the RNA polymerase comprises a T3 RNA polymerase, a T7 RNA polymerase, a SP6 RNA polymerase, or a combination thereof. In an aspect, the RNA polymerase comprises a T7 RNA polymerase. An average concentration for T7 RNA polymerase is 5 units of enzyme/microliter of reaction mixture.

In an aspect, the transcription reaction mixture comprises Mg⁺⁺ ions. The optimal concentration of Mg⁺⁺ ions in the reaction mixture is determined by the total NTP concentration in the reaction mixture. In an aspect, the Mg⁺⁺ ions are provided in the form of MgCl₂ or Mg(OAc) 2. In an aspect, the free Mg⁺⁺ concentration in the reaction mixture is from 1 millimolar (mM) to 100 mM, 1 to 50 mM, 1 to 25 mM, or 1 to 10 mM.

The transcription mixture also includes a capping reagent, also referred to as a capping enzyme, which catalyzes the in vitro addition of a cap nucleotide (cap structure) to the 5′ end of the mRNA molecule. The cap structure is involved in pre-mRNA processing and splicing, mRNA export, translation initiation, and mRNA stability, and is attached by a 5′ to 5′ triphosphate linkage to the first base of the RNA molecule. The cap structure can be a guanine triphosphate, added to the 5′ end of the mRNA molecule, and can be methylated (e.g., monomethylated, demethylated or trimethylated). Examples of the cap structure include a 7-methylguanosine triphosphate (m7GpppN, where N is the terminal 5′ nucleotide of the RNA molecule), a 2,7-methylguanosine triphosphate (m2′7GpppN), and 2,2,7-trimethylguanosine triphosphate (m2,2,7GpppN). The source and/or type of the capping reagent is not particularly limited, and examples include Vaccinia Capping System (New England Biolabs), CleanCap® (TriLink Biotechnologies), and ScriptCap® m7G Capping System (Cellscript, Madison, Wis., USA) In an aspect, the capping reagent is CleanCap®. The capping can be co-transcriptional or post-transcriptional. In an aspect, the capping is co-transcriptional.

The transcription mixture can also include a reducing agent to keep the RNA polymerase in its active state. In an aspect, the reducing agent includes dithiothreitol (DTT), dithioerythritol (DTE), Tris(2-carboxyethyl) phosphine (TCEP), β-mercaptoethanol, or a combination thereof. In an aspect, the concentration of the reducing reagent is 1 to 50 mM, 1 to 40 mM, 1 to 30 mM, or 1 to 20 mM, or 1 to 10 mM.

The transcription mixture can further include an enzyme to remove an inhibitory reaction product from the transcription reaction product. In an aspect, the transcription mixture includes a pyrophosphorylase to remove pyrophosphate, a reaction product generated during the in vitro transcription.

The contacting of the DNA template and the in vitro transcription mixture is a continuous process performed under conditions for in vitro transcription (RNA synthesis) of the sequence encoding the open reading frame. During the continuous synthesis of the RNA molecule, the components of the transcription reaction mixture are gradually depleted. The sensor is in communication with the bioreactor and measures the concentration of each of the reactants in the transcription mixture. In an aspect, the sensor is an inline UV probe (NIRRIN Technologies) and continuously measures the concentration of each reactant in the bioreactor. Based upon the signals received from the sensor, the control module determines whether one or more of the concentrations is above, at, or below a threshold amount needed to transcribe the DNA template to RNA. The threshold amount is determined through the data collected by the sensor in combination with a feedback control algorithm. When the concentration falls below a predetermined threshold level, the control module feeds the reactant to the bioreactor. The control module thus maintains the reactants at defined concentrations using input received by the sensor. Rate calculations in the control algorithm are also used to determine the half-life of the RNA polymerase, which is added as needed.

The bioreactor is configured to include at least one supply line that supplies a reactant to the bioreactor. The control module activates a pump to move the reactant from a supply reservoir, through the supply line, and into the bioreactor. The bioreactor also includes an outlet which connects to the mRNA separation system. The bioreactor further includes a mechanism for stirring the contents in the bioreactor, and which can be operated at variable stirring rates. The bioreactor may be thermally regulated to maintain a predetermined specific temperature. In an aspect, the temperature of the bioreactor is 30° C. to 40° C., or 37-56° C. for a high temperature RNA polymerase.

The removal of the RNA molecule from the bioreactor is necessary for continuous mRNA synthesis. To facilitate removal of the RNA molecule from the transcription reaction product, an affinity tag can be incorporated at a 3′ end of the RNA molecule and the transcription reaction product fed through an affinity column comprising an immobilized ligand for the affinity tag. In an aspect, the RNA molecule produced by the in vitro transcription reaction includes an affinity tag at a 3′ end, and is thus an affinity-tagged RNA molecule. The sequence for the affinity tag can be encoded in the DNA template such that during the in vitro transcription reaction, the affinity tag is bound to the 3′ end of the open reading frame. The affinity tag can be a polyadenylate tail (poly(A) tail), a poly(histidine) tail, or a combination thereof. In an aspect, the affinity tag is a poly(A) tail. The length of the poly(A) tail is not limited, and can be any length that is capable of effectively binding to the immobilized ligand. In an aspect, the affinity tag is a poly(histidine) tail. The poly(histidine) tail can comprise, for example, six or more consecutive histidine residues.

The transcription reaction product is continuously fed (flows) from the bioreactor to the RNA separation system, and specifically, to the at least one affinity column in the capture module. The affinity column comprises the immobilized ligand, and as the transcription reaction product passes through the affinity column, the affinity-tagged RNA molecule binds to the immobilized ligand. The binding between the immobilized ligand in the affinity column and the affinity-tagged RNA molecule advantageously occurs under conditions that do not affect the reactants and without the need to change the reaction conditions in the reaction module. In contrast, separation methods such as poly dT columns require the use of a high salt concentration to facilitate binding to mask the effects of phosphate repulsion. However, these high salt concentrations have a negative effect on the reaction conditions in the bioreactor, making it difficult to recycle any unused reactants back into the system. In addition, anion exchange chromatography alone is unable discriminate, under reactor operating conditions, between the reactants in the transcription mixture and the produced RNA molecule.

In an aspect, the immobilized ligand comprises a polyA binding protein (PABP), a poly(histidine) PSBP, engineered lysine, engineered cysteine, or a combination thereof. In an aspect, the immobilized ligand comprises PABP. PABP is a stable structure composed of a four-stranded, antiparallel B sheet backed by two a helices. PABP binds to the poly(A) tail on the 3′ end of an mRNA molecule, and is highly conserved across eukaryotic species. PABP is composed of four RNA recognition motifs (RRM): RRM1, RRM2, RRM3, and RRM4. In particular, RRM1 and RRM2 are associated with the binding to the mRNA poly(A) tail. Thus in an aspect, the immobilized ligand comprises a poly(A) binding protein and affinity tag of the affinity-tagged RNA molecule is a poly(A) tail.

In an aspect, the PABP is a full-length protein. In an aspect, the PABP comprises truncated BABP. The truncated PABP can be produced in E. coli using standard industry practices designed to promote facile immobilization. The source of the PABP is not limited. In an aspect, the PABP is from Saccharomyces cerevisiae (GenBank: EDN59171.1). In an aspect, the truncated PABP comprises regions RRM1 and RRM2 of the protein. In an aspect, the ligand comprises truncated PABP which has been modified to include a cysteine sulfhydryl group at the carboxyl end of the truncated protein.

In an aspect, the affinity column comprises activated beads as a substrate for the immobilization of the ligand. The ligand can be immobilized on the surface of the activated beads either before or after loading of the beads into the affinity column. In an aspect, the beads are activated beads including a functional group on a surface. The functional group binds to an reactive group present on the ligand and thus facilitates the immobilization of the ligand to the bead. Such beads are known in the art. For example, the functional group can be an epoxy (oxirane) group. The immobilization of the ligand to the beads can be performed at a pH of 6.5 to 8.5 to facilitate reaction between the functional group on the beads and a reactive group of the ligand. Alternatively, the affinity column can be a monolithic column (e.g., Convective Interaction Media, CIM®; Sartorius) having an affinity for the RNA molecule. Similar to beads, the columns can be functionalized with epoxy groups or utilize chelate chemistry. Surfaces of the monolith can be grafted with different functionalities as needed, such as hydrophobic and ion exchange moieties, to facilitate mRNA binding. In such cases, the ligand is a functional group on surfaces of the monolith.

Binding of the affinity-tagged RNA molecule to the ligand in the affinity column occurs until a predetermined amount of the affinity-tagged RNA molecule bound to the affinity column has been reached. In an aspect, the binding of the affinity-tagged RNA molecule to the ligand in the affinity column occurs until the loading capacity of the affinity column has been reached. The binding capacity of the affinity column can be based upon, for example, the flow conditions, the amount of ligand in the affinity column, the concentration of RNA molecules in the transcription reaction product, etc. Upon reaching the desired level of loading, the bound affinity-tagged RNA molecule is eluted from the affinity column. The binding of the affinity-tagged RNA molecule to the affinity column is monitored using the sensor in the control module. In an aspect, the capture modules includes at least two affinity columns (e.g., first and second affinity columns). When the sensor signals that the desired level of binding of the affinity-tagged RNA molecule in the column has been reached, the control module instructs the RNA separation system to divert the flow of the transcription reaction product from the first affinity column to the second affinity column. In an aspect, bound RNA is eluted from the first affinity column as transcription reaction product flows from the bioreactor to the second affinity column. Once the desired level of loading of the affinity-tagged RNA molecules in the second affinity column has been achieved, the control module switches the flow of the transcription reaction product back to the first affinity column while the bound RNA is eluted from the second affinity column. Following elution, the offline column is washed and equilibrated to prepare for the next loading. Accordingly, the transcription reaction product is continuously pumped through the capture module.

Advantageously, the binding of the affinity-tagged RNA to the ligand in the affinity column can be performed under physiological conditions. For example, the binding can occur without the need for a high salt concentration and can occur at a physiological pH of 6.5 to 8.2, or 6.8 to 8, or 7-8. The bound RNA can be eluted from the affinity column using any suitable eluent.

The material eluted from the affinity columns (eluate) flows to the purification module including the continuous chromatography column, where it is subjected to polishing and buffer formulation. In the purification module, contaminants or impurities contained in the eluate, for example, double stranded RNA (dsRNA), bind to the material of the column and are separated from the RNA molecules. Thus, the material exiting the purification module is substantially pure RNA. In an aspect, the flow through chromatography column comprises a cellulose material. The purity of the purified RNA is 90%, 95%, 97%, 98%, 99%, or 100%. Depending upon the impurity profile, additional steps may be taken to further reduce any remaining impurities to optimum levels. The purification modules also allows for buffer formulation. In an aspect, the purified RNA is combined with a buffer in the purification module.

In an aspect, the purified RNA molecule includes an open reading frame encoding a protein of interest. In an aspect, the open reading frame encodes an antigen of a virus, a bacteria, a parasite, a cancer cell, or a combination thereof. The antigen can be a surface protein or a non-surface protein.

The virus can be a pathogenic virus, examples of which include cytomegalovirus (CMV), coxsackie virus, Crimean-Congo hemorrhagic fever virus, chikungunya virus, dengue virus, Dhori virus, Eastern equine encephalitis (EEE) virus, ebola virus, Epstein Barr virus (EBV), hepatitis virus, herpesvirus, human immunodeficiency (HIV) virus, human papilloma virus, human SARS corona virus, SARS COV-2, human T lymphotropic virus (HTLV), influenza virus, Japanese encephalitis virus, measles virus, mumps virus, poliovirus, Norwalk virus, smallpox, rabies virus, reovirus, rotavirus, rubella virus, severe fever with thrombocytopenia syndrome (SFTS) virus, respiratory syncytial virus (RSV), varicella zoster virus, Western equine encephalitis virus, West Nile virus, yellow fever virus, Zika virus, or a combination thereof.

The bacteria can be a pathogenic bacteria, examples of which include Bacillus sp., Baronella sp., Bordatella sp., Borelli asp., Brucella sp., Campylobacter sp., Chlamydia sp., Clostridium sp., Corynebacterium sp., Enterococcus sp., Escherichia sp., Haemophilis sp., Helicobacter sp., Legionella sp., Leptospira sp., Listeria sp., Mycobacterium sp., Mycoplasma sp., Neisseria sp., Rickettsia sp., Pseudomonas sp., Salmonella sp., Shigella sp., Staphylococcus sp., Streptococcus sp., Treponema sp., Vibrio sp., Yersinia sp., or a combination thereof.

The parasite can be a pathogenic parasite, examples of which include Acanthamoeba spp., Balamuthia spp., Babesia sp., Balantidium coli, Blastocystic sp., Cryptospiridium sp., Cyclospora cayetanensis, Entamoeba histolytica, Giardia lamblia, Isospora bello, Leishmania sp., Naegleria foweri, Plasmodium sp., Rhinosporidium seeberi, Sarcocystis sp., Toxoplasma gondii, Trichomonas sp., Trypanosoma sp., or a combination thereof

The cancer cell can be, for example, an animal cancer cell or a human cancer cell.

This disclosure is further illustrated by the following examples, which are non-limiting.

### Examples

### Example 1: Continuous mRNA Production Apparatus

The continuous mRNA production apparatus includes a controlled reactor and a mRNA separation device (see FIG. 1). An inline sensor (e.g., a UV/NIR probe; NIRRIN Technologies) will measure reactant concentrations. A feedback control algorithm will maintain reactants at defined concentrations using UV probe inputs. Rate calculations, in the control algorithm will be used to determine the half-life of the RNA polymerase (e.g., T7), which will be added as needed. The reaction mixture will be continuously pumped through the mRNA binding columns. Differential measurements, utilizing the NIRRIN inline probe, will be used to change columns at maximum loading. The offline column will be washed, eluted, and equilibrated to prepare for the next loading. Eluted material will flow to the continuous chromatography system for polishing and buffer formulation.

### Example 2: mRNA Purification Device

We will produce an mRNA binding column utilizing immobilized poly(A)-binding protein (PABP). We will produce truncated PABP in E. coli using standard industry practice engineered to promote facile immobilization. We will use S. cerevisiae's sequence as a starting template (GenBank: EDN59171.1). In order to simplify immobilization we will introduce a new cysteine residue in the carboxyl end of the truncated protein. Immobilization at pH 7-8 will react with the cysteine sulfhydryl group leaving other potential groups unreactive. The amount of protein sequence remaining between the mRNA binding region (RRM1 and RRM2) will be varied to determine optimal immobilization, mRNA binding, and elution. The purified protein will be covalently bonded to both activated (oxirane) beads and monolith and compared for effectiveness

### Example 3: Control Strategies

Control strategies will be built that utilize biosensor signals in an algorithm for overall feedback control. A reaction mechanism-based model will be first established to guide the design, control and optimization of the integrated continuous reaction and separation process. We will start the modeling from the very basic reaction stoichiometry for net synthesis of an RNA transcript consisting of n nucleotide and develop a rate equation:

N_{A}ATP+n_{C}CTP+n_{G}GTP+n_{U}UTP→RNAₙ+(n−1)PPi+DNA

A reaction kinetic model and reactor operating equations will be adjusted based on the following information.
The exact chemistry will be adjusted based our new reaction system. For example, the rate equation may be adjusted by considering a fifth base, pseudo-uridine
The T7 polymerase will have a half-life. We can infer the half-life from the rate of consumption of the bases, and then used to modify the rate equation.
The rate equation may further be adjusted by considering the use of CleanCap from TriLink as the 5′capping agent, which has been shown to be efficient in initiating the transcription.

By using continuous inline measurements, we will optimize the continuous manufacturing conditions such as the initial NTP concentrations, the continuous RNA removal rate by the binding column, and the feed/withdrawing rates, so that steady-state continuous RNA production with an optimal conversion of NTPs for a highest productivity of RNA can be achieved.

### Example 4: At-line Photo-Electric ELISA Solid State mRNA PABP Sensor

We will develop mRNA sensors based on a photo-electric ELISA solid state, platform technology (Advanced Silicon Group) with PABP (FIGS. 2A-B). The mRNA sensor signal will feed into the mRNA control algorithm for feedback control.

### Example 5: E. coli Expression of Truncated PABP and Immobilization of PABP

A truncated yeast PABP was cloned into E. coli (BL-21 (DE-3)) with pET 24a utilizing an encoded poly histidine 3′ tail. One ml of overnight culture (LB Miller, 30 μg/ml Kan) was used to inoculate 50 mls of pre-warmed (30° C.) media (LB Miller, 30 μg/ml Kan), grown to OD₆₀₀ of 0.2 and induced with 1 mM IPTG and incubated for an additional 2 hours.

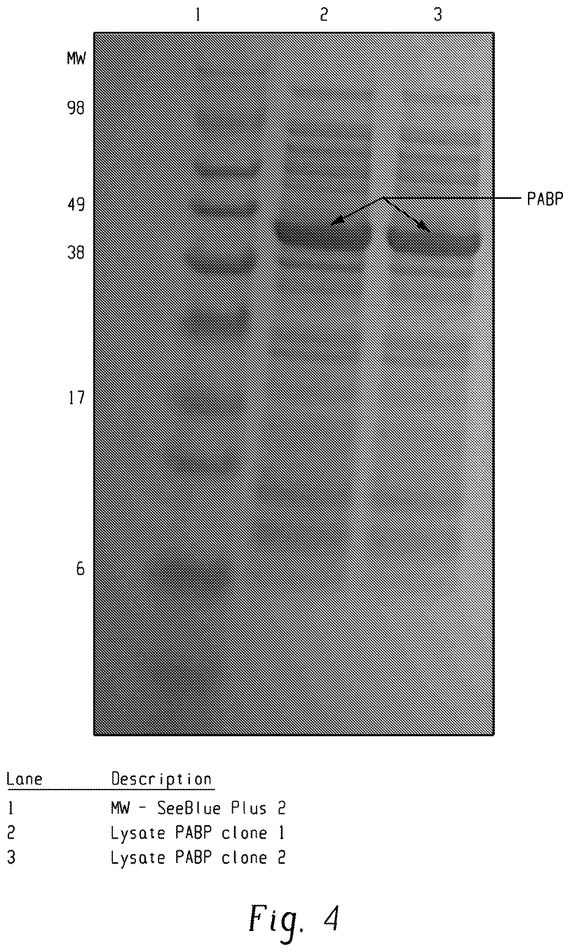

The cells were harvest by centrifugation and the pellets suspended in buffer (20 mM Tris, 150 mM NaCl, pH 7.5) and lysed by sonication. FIG. 4 shows the PAGE analysis of polyadenine binding protein expression.

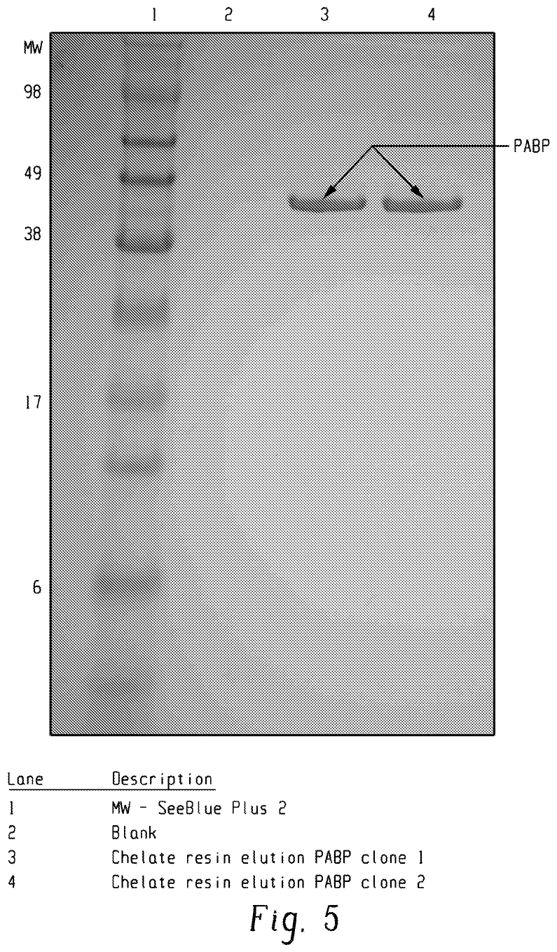

A 0.2 ml column of EMD Chelate (M) was charged with Co²⁺, rinsed with 5 CV of water and followed by 5 CV of 500 mM NaCl. The column was then equilibrated with 5 CV of equilibration buffer (20 mM Tris, 150 mM NaCl, pH 7.5). PABP lysate (0.5 ml) was loaded onto the column. The column was washed/equilibrated with 5 CV of 20 mM Tris, 150 mM NaCl, pH 7.5. FIG. 5 shows the PAGE analysis of EMD Chelate PABP Purification.

### Example 6: Capture and Purification of mRNA

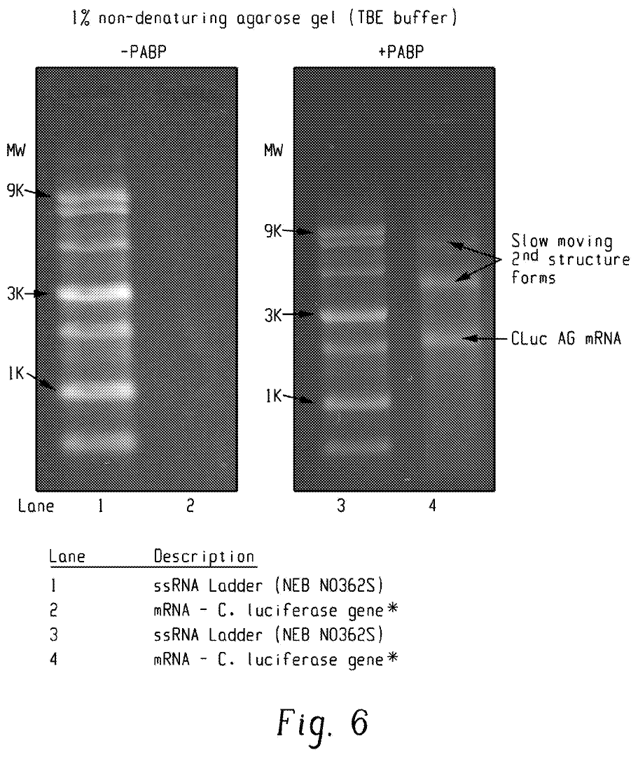

mRNA synthesis was carried out with NEB's E2080S, HiScribe™ T7 mRNA Kit with CleanCap® Reagent AG utilizing the cut control plasmid CLuc AG. After 2 hours of mRNA synthesis, the reaction mixture (20 μl diluted to 0.2 ml with equilibration buffer) was transferred to the immobilized PABP column (step 2). The column was washed with 5 CV of equilibration buffer. Purified mRNA was eluted with elution buffer (5 M urea, 20 mM Tris, 150 mM NaCl, pH 7.5). The eluate was analyzed by electrophoresis in a 1% non-denaturing agarose gel (TBE buffer). FIG. 6 shows the purification of the mRNA.

The compositions, methods, and articles can alternatively comprise, consist of, or consist essentially of, any appropriate materials, steps, or components herein disclosed. The compositions, methods, and articles can additionally, or alternatively, be formulated so as to be devoid, or substantially free, of any materials (or species), steps, or components, that are otherwise not necessary to the achievement of the function or objectives of the compositions, methods, and articles.

All ranges disclosed herein are inclusive of the endpoints, and the endpoints are independently combinable with each other (e.g., ranges of “up to 25 wt. %, or, more specifically, 5 wt. % to 20 wt. %”, is inclusive of the endpoints and all intermediate values of the ranges of “5 wt. % to 25 wt. %,” etc.). “Combinations” is inclusive of blends, mixtures, alloys, reaction products, and the like. The terms “first,” “second,” and the like, do not denote any order, quantity, or importance, but rather are used to distinguish one element from another. The terms “a” and “an” and “the” do not denote a limitation of quantity and are to be construed to cover both the singular and the plural, unless otherwise indicated herein or clearly contradicted by context. “Or” means “and/or” unless clearly stated otherwise. Reference throughout the specification to “some embodiments”, “an embodiment”, and so forth, means that a particular element described in connection with the embodiment is included in at least one embodiment described herein, and may or may not be present in other embodiments. In addition, it is to be understood that the described elements may be combined in any suitable manner in the various embodiments. A “combination thereof” is open and includes any combination comprising at least one of the listed components or properties optionally together with a like or equivalent component or property not listed

Unless specified to the contrary herein, all test standards are the most recent standard in effect as of the filing date of this application, or, if priority is claimed, the filing date of the earliest priority application in which the test standard appears.

Unless defined otherwise, technical and scientific terms used herein have the same meaning as is commonly understood by one of skill in the art to which this application belongs. All cited patents, patent applications, and other references are incorporated herein by reference in their entirety. However, if a term in the present application contradicts or conflicts with a term in the incorporated reference, the term from the present application takes precedence over the conflicting term from the incorporated reference.

While particular embodiments have been described, alternatives, modifications, variations, improvements, and substantial equivalents that are or may be presently unforeseen may arise to applicants or others skilled in the art. Accordingly, the appended claims as filed and as they may be amended are intended to embrace all such alternatives, modifications variations, improvements, and substantial equivalents.

1. A modular apparatus for continuous production of purified RNA, the apparatus comprising, in operable communication:
a reaction module comprising a bioreactor configured for continuous synthesis of an affinity-tagged RNA molecule by in vitro transcription using reactants in an in vitro transcription mixture;
a control module comprising a sensor; and
an RNA separation system configured to receive a transcription reaction product comprising the affinity-tagged RNA molecule from the bioreactor and to separate the affinity tagged RNA molecule from the transcription reaction product,
wherein the RNA separation system comprises a capture module comprising an affinity column for binding the affinity-tagged RNA molecule and a purification module comprising a continuous chromatography column,
wherein the control module controls input of the reactants to the bioreactor and monitors separation of the affinity-tagged RNA molecule using the sensor, and
wherein the sensor is in communication with the reaction module and the RNA separation system and the sensor provides feedback control of the transcription mixture in the reaction module and the transcription reaction product in the RNA separation system.

2. The apparatus of claim 1, wherein the affinity-tagged RNA molecule is an mRNA molecule.

3. The apparatus of claim 1, wherein the affinity column comprises immobilized poly(A)-binding protein and the affinity tag is a poly(A) tail or wherein the affinity column comprises immobilized poly histidine PSBP and the affinity tag is a poly-histidine tail.

4. (canceled)

5. The apparatus of claim 1, wherein the sensor comprises a plurality of sensors that independently monitor nucleotides, capping molecules, RNA polymerase, magnesium levels, or a combination thereof.

6. The apparatus of claim 1, wherein the continuous chromatography column removes an impurity from the affinity-tagged RNA molecule received from the at least one affinity column.

7. The apparatus of claim 1, further comprising
a first supply line that supplies a reactant for in vitro transcription to the bioreactor, wherein the first supply line is connected to a reservoir comprising the at least one reactant; or
a plurality of supply lines each of which independently supplies a reactant for in vitro transcription to the bioreactor; or
a recirculation line from an output of the affinity column to an input of the bioreactor; or
a first transfer line from an outlet of the reaction module to an inlet of the capture module and a second transfer line from an outlet of the capture module to an inlet of the purification module, wherein the capture module comprises a plurality of affinity columns each including an inlet and an outlet, and the outlet of each of the plurality of affinity columns is connected to the second transfer line; or
a second supply line that supplies an elution buffer to the at least one affinity column.

8.-13. (canceled)

14. A method for continuous production of purified RNA, the method comprising:
contacting a DNA template comprising a sequence encoding an open reading frame and a sequence encoding an affinity tag with reactants in an in vitro transcription mixture, and under conditions suitable to provide a transcription reaction product comprising an affinity-tagged RNA molecule, wherein the contacting is performed in a bioreactor operating in continuous mode;
feeding the transcription reaction product to an affinity column comprising an immobilized ligand and binding the affinity-tagged RNA molecule to the immobilized ligand;
eluting the bound affinity-tagged RNA molecule from the affinity column;
passing the eluted affinity-tagged RNA molecule through a continuous chromatography column to provide the purified RNA; and
providing feedback control of the in vitro transcription mixture in the bioreactor and the transcription reaction product in the affinity column.

15. The method of claim 14,
wherein the affinity-tagged RNA molecule is an mRNA molecule; or
wherein the immobilized ligand comprises a poly(A) binding pattern protein and affinity tag of the affinity-tagged RNA molecule is a poly(A) tail, wherein the immobilized ligand comprises a poly histidine PSBP and the affinity tag is a poly-histidine tail, wherein the immobilized ligand comprises lysine and the affinity tag is or a combination thereof; or
wherein the poly(A) binding protein comprises a full length protein or a truncated protein.

16. The method of claim 15, wherein each of the contacting, adding, binding, eluting, passing, and providing are continuous processes.

17. The method of claim 14, wherein feedback control of the in vitro transcription mixture in the bioreactor comprises monitoring a concentration of at least one reactant in the in vitro transcription mixture during the contacting and determining if the concentration is above, at, or below a threshold level.

18. The method of claim 17, further comprising supplying a reactant to the bioreactor when the concentration falls below a threshold level.

19. The method of claim 17, wherein the feedback control of the in vitro transcription mixture in the bioreactor is performed by an inline sensor that is in communication with the bioreactor and that detects a concentration of at least one reactant in the in vitro transcription mixture.

20. The method of claim 14, wherein feedback control of the transcription reaction product in the affinity column comprises monitoring the binding of the affinity-tagged RNA molecule to the immobilized ligand using an inline sensor in communication with the affinity column, wherein the eluting of the bound affinity-tagged RNA molecule from the affinity column begins when a predetermined amount of the affinity-tagged RNA molecule bound to the affinity column is reached.

21. (canceled)

22. The method of claim 14, wherein the DNA template encodes a promoter operably linked to the sequence encoding the open reading frame and the sequence encoding the affinity tag.

23. The method of claim 14, wherein the contacting is performed under conditions suitable for in vitro transcription.

24.-26. (canceled)

27. The method of claim 14, wherein the in vitro transcription mixture comprises guanosine-5′-triphosphate, adenosine triphosphate, cytidine triphosphate, uridine triphosphate, pseudouridine triphosphate, dihydrouridine triphosphate, 4-thiouridine, inosine triphosphate, 7-methylguanosine triphosphate, 2,7-dimethylguanosine triphosphate, 2,2,7-trimethylguanosine triphosphate, queuosine triphosphate, a capping reagent, magnesium ions, RNA polymerase, a buffer, or a combination thereof.

28. The method of claim 14, wherein the affinity column comprises a first affinity column and a second affinity column each comprising the immobilized ligand, wherein the feeding of the transcription reaction product to the first affinity column occurs until a predetermined amount of the affinity-tagged RNA molecule is bound to the first affinity column and then feeding of the transcription reaction product switches to the second affinity column; wherein the bound affinity-tagged RNA molecule is elated from the first affinity column while the transcription reaction product is fed to the second affinity column.

29. (canceled)

30. The method of claim 14, wherein the binding of the affinity-tagged RNA molecule to the immobilized ligand occurs at pH of 6.5 to 8.5.

31. The method of claim 14, wherein the continuous chromatography column comprises a cellulose material.

32. The method of claim 15, wherein the purified RNA comprises an open reading frame encoding an antigen of a virus, a bacteria, a parasite, a cancer cell, or a combination thereof.
